(19) United States
(12) Patent Application Publication
(10) Pub. No.: US 2019/0328305 A1
(43) Pub. Date: Oct. 31, 2019

(54) SYSTEM AND METHOD FOR TESTING A CONDITION OF THE NERVOUS SYSTEM USING VIRTUAL REALITY TECHNOLOGY

(71) Applicants: Carl Zeiss Meditec, Inc., Dublin, CA (US); Carl Zeiss Meditec AG, Jena (DE)

(72) Inventors: Robert J. WOOD, Naples, FL (US); Matthias MONHART, Winterthur (CH); Maximilian STOCKER, Donzdorf (DE)

(73) Assignees: Carl Zeiss Meditec, Inc., Dublin, CA (US); Carl Zeiss Meditec AG, Jena, CA (DE); Carl Zeiss Meditec AG, Jena, CA (DE)

(21) Appl. No.: 16/068,039

(22) PCT Filed: Jan. 18, 2017
(86) PCT No.: PCT/EP2017/050959

§ 371 Date: Jul. 3, 2018

Related U.S. Application Data
(60) Provisional application No. 62/281,437, filed on Jan. 21, 2016.

(51) Int. Cl. A61B 5/00; A61B 3/08; G06F 3/01; G02B 27/00; G02B 27/01; G06T 19/00; G16H 80/00

(52) U.S. Cl. CPC A61B 5/40; A61B 3/08; G06F 3/013; G16H 80/00; G02B 27/0093; G02B 27/0172; G06T 19/006; G06F 3/017

(57) ABSTRACT

A system and a method for testing a condition of a subject's nervous system using virtual reality technology are described. The method includes displaying a visual stimulus to the subject in a virtual reality environment. Eye and body movements of the subject are tracked as the subject focuses on the visual stimulus. The body movements may include head movements. Based on the eye and body movements, the condition of the subject's nervous system is evaluated and then results of the evaluation describing the subject's nervous system condition is reported to a user, such as a clinician, for further analysis thereof.

### PRIORITY

This application claims priority to U.S. Provisional Application Ser. No. 62/281,437 filed Jan. 21, 2016, the contents of which are hereby incorporated by reference.

### BACKGROUND

Measuring a condition of a person's nervous system allows one to establish a baseline, study specific behavior of the person, and analyze changes resulting from any past incidences of head injuries, alcohol and/or drug intake, etc. The person's ability to perform a task after going through such incidences—be it driving a car after intake of alcohol or going back to the field after a traumatic brain injury (also referred to as concussion), collision, and/or clash during a training or game—are questions that needs to be answered immediately and sometimes locally to avoid further damage to the person's nervous system. Therefore, quick and objective testing at the place of an event is a highly desirable requirement.

Existing systems and/or methods for testing a person's nervous system are based on eye/gaze tracking. However, eye tracking is prone to errors and is not an accurate and reliable way to completely test a condition of the person's nervous system. Furthermore, existing systems and/or methods require an expert or person skilled in the area to perform the testing. Such experts may not be available at all times to perform a test especially when the test needs to be performed right after an incident or accident has occurred.

Thus, there is a need for a portable testing system and a method for testing a condition of a person's nervous system, which is easy to use by even an ordinary person and is capable of testing the condition completely and accurately. Here we describe such a portable testing system and a method that is capable of testing a person and evaluating the condition of the person either locally or remotely based on the eye and body movements of a person using virtual reality technology.

### SUMMARY

According to one aspect of the subject matter described in the present application, a system for testing a condition of a subject's nervous system using virtual reality technology includes a virtual reality headset for displaying a visual stimulus separately to each eye of the subject in a virtual reality environment; an eye sensor for tracking eye movements as the subject focuses on the visual stimulus; a motion sensor for tracking body movements as the subject focuses on the visual stimulus in the virtual reality environment; and a processor for evaluating the condition of the subject's nervous system based on the eye and the body movements, wherein results of the evaluation are stored or transmitted for display to a clinician.

According to another aspect of the subject matter described in the present application, a method for testing a condition of a subject's nervous system using virtual reality technology includes displaying a visual stimulus to the subject in a virtual reality environment; tracking eye and body movements of the subject as the subject focuses on the visual stimulus; evaluating the subject's nervous system condition based on the eye and body movements of the subject; and reporting the subject's nervous system condition to a clinician for further analysis thereof.

Further aspects include various additional features and operations associated with the above and following aspects and may further include, but are not limited to corresponding systems, methods, apparatus, and computer program products.

The features described herein are not all-inclusive and many additional features will be apparent to one of ordinary skill in the art in view of the figures and description. Moreover, it should be noted that the language used in the specification has been principally selected for readability and instructional purposes and not to limit the scope of the inventive subject matter.

### BRIEF DESCRIPTION OF THE DRAWINGS

FIG. 1 is a block diagram of an exemplary system architecture for testing a condition of a subject's nervous system using virtual reality technology.

FIG. 2 is a flowchart of an example method for locally testing and evaluating a condition of the nervous system using virtual reality technology.

FIGS. 3A and 3B are flowcharts of an example method for testing a subject and remotely evaluating a condition of the subject's nervous system.

FIGS. 4A and 4B depict a front view and a top view of the virtual reality headset discussed in the present invention, respectively.

FIG. 5A depicts an example visual stimulus that is displayed separately to each eye of a subject (e.g., individually adapt the focal position to the physiology of each eye separately) using the virtual reality headset. FIG. 5B depicts the visual stimulus that is displayed separately to each eye as moving towards each other in order to identify the subject's nasal field of stereopsis.

FIG. 6 depicts an example task that may be instructed to be performed by a subject in order to test the subject's nervous system condition.

FIG. 7 depicts an example message indicating the subject's nervous system condition to a clinician.

FIG. 8 is a block diagram of an example computing device, which may be representative of a portable concussion tester, a clinician's device, and/or or a server.

### DETAILED DESCRIPTION

Example System Architecture

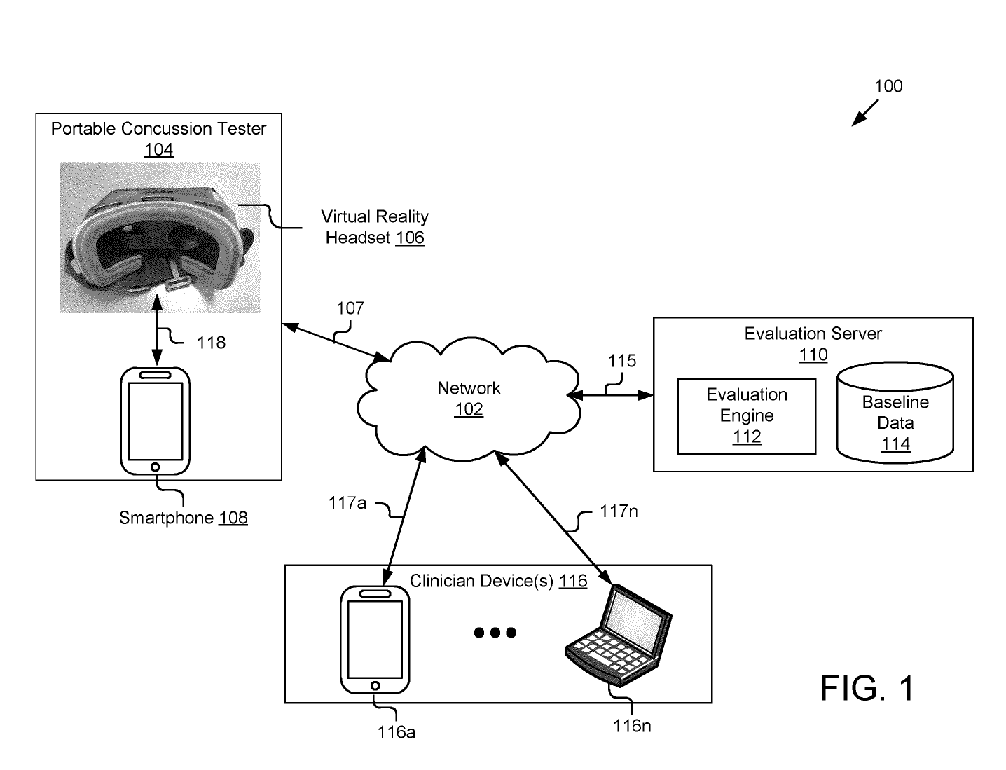

FIG. 1 is a block diagram of an exemplary system architecture 100 for testing a condition of the nervous system using virtual reality technology. In the depicted embodiment, the system architecture 100 includes a portable concussion tester 104 (including a virtual reality headset 106 and a smartphone 108), an optional evaluation server 110, and one or more clinician devices 116a through 116n (individually or collectively referred to herein as 116). These entities of the system architecture 100 are communicatively coupled via a network 102. The evaluation server 110 is optional since the need of the server 110 depends upon whether evaluation of a subject after performing a test using the portable concussion tester 104 needs to be performed locally by the tester 108 itself or on the server 110. If the decision is to evaluate the test remotely, data from the portable concussion tester 104 is sent to the evaluation server 110 via the network 102 for evaluation or processing purposes. It should be understood that the present disclosure is not limited to this configuration and a variety of different system environments and configurations may be employed and are within the scope of the present disclosure.

The network 102 may be of a conventional type, wired or wireless, and may have numerous different configurations including a star configuration, token ring configuration, or other configurations. Furthermore, the network 102 may include a local area network (LAN), a wide area network (WAN) (e.g., the Internet), and/or other interconnected data paths across which multiple devices may communicate. In some embodiments, the network 102 may be a peer-to-peer network. The network 102 may also, be coupled to or include portions of a telecommunications network for sending data in a variety of different communication protocols. In some embodiments, the network 102 includes Bluetooth™ communication networks or a cellular communications network for sending and receiving data including via short messaging service (SMS), multimedia messaging service (MMS), hypertext transfer protocol (HTTP), direct data connection, WAP, email, etc. In addition, although FIG. 1 illustrates a single network 102 coupled to the portable concussion tester 104 including the virtual reality headset 106 and the smartphone 108, the evaluation server 110, and the one or more clinician devices 116 that are illustrated, in practice, one or more networks 102 may be connected to these entities.

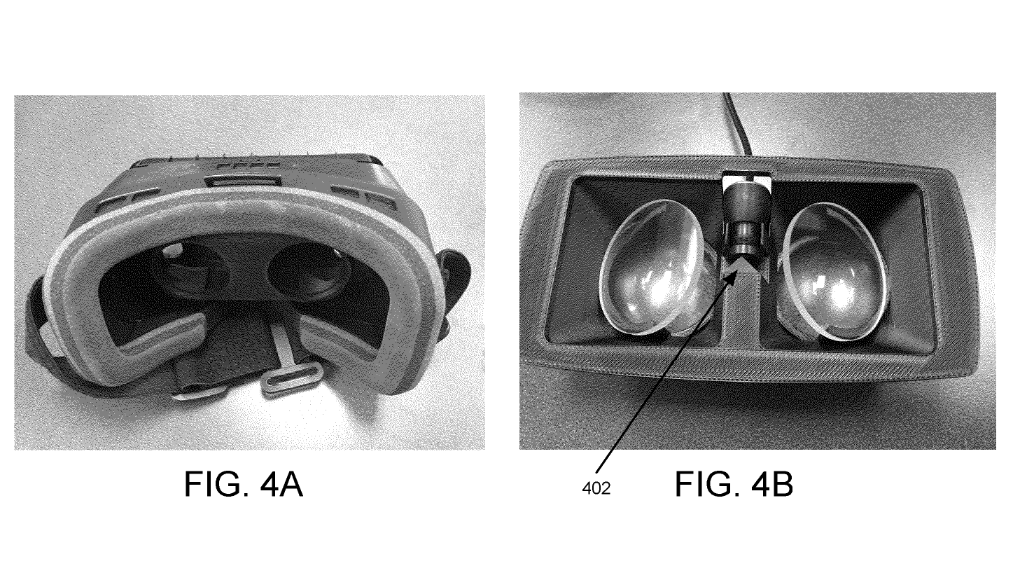

The portable concussion tester 104 is any portable testing equipment or device that is capable of testing the condition of a subject's nervous system on the go. For example, the portable concussion tester 104 is capable of testing the subject after he/she has gone through a potentially traumatic collision/clash or mild traumatic brain injury (i.e., concussion) during a training or game. The portable concussion tester 104 can be used either by the subject themselves, a coach, or a first responder to the accident. In some embodiments, the portable concussion tester 104 discussed herein can be used to chart/monitor/track the progression of a subject's cognizant awareness over certain time periods to determine neurological conditions relating to Alzheimer's, Dementia, and any age or degenerative related disease states. In some embodiments, the portable concussion tester 104 may be capable of performing some of the physical assessment exams or tests known in the art. Once a test is performed by the portable concussion tester 104, the tester 104 may process the test to evaluate the condition of the subject's nervous system either locally or remotely with the help of the evaluation server 110. As depicted, the portable concussion tester 104 includes a virtual reality headset 106, which is a wearable headset that lets a wearer to immerse in a virtual reality environment. In some embodiments, the virtual reality headset 106 may be further capable of providing an augmented reality experience to the wearer where one or more images/visual stimulus are superimposed on the wearer's view of the real world. The virtual reality headset 106 covers both eyes similar to a pair of ski goggles. FIGS. 4A and 4B show a front view and a top view of an exemplary virtual reality headset, respectively that may be used according to one aspect of the present invention. As shown in FIG. 4B, the virtual reality headset 106 may use a prism (indicated by reference numerals 402) to split the light path so that both eyes can be observed on a single camera, despite the separate optical paths between each eye and their visible portion of the display. In order to introduce the optical camera path into the “light signal” path without disrupting the latter, hot mirrors, which enables infrared (IR) light to be mirrored while visible light to be not mirrored, can be used in some implementations. The virtual reality headset 106 works in conjunction with a smartphone, such as the smartphone 108, to test a condition of a subject's nervous system. In a preferred embodiment, the smartphone 108 is positioned or inserted into the virtual reality headset 106 such that the display of the smartphone 108 faces the wearer's eyes. In some embodiments, the virtual reality headset 106 and the smartphone 108 may be locally connected with each other (as indicated by reference numeral 118) using Bluetooth™, Infrared (IR) connection, or any standard means of communication known in the art for local connections. As depicted, the portable concussion tester 104 (including the virtual reality headset 106 and the smartphone 108) is connected to the network 102 via signal line 107 for communicating with one or more clinician devices 116a through 116n and/or the evaluation server 110.

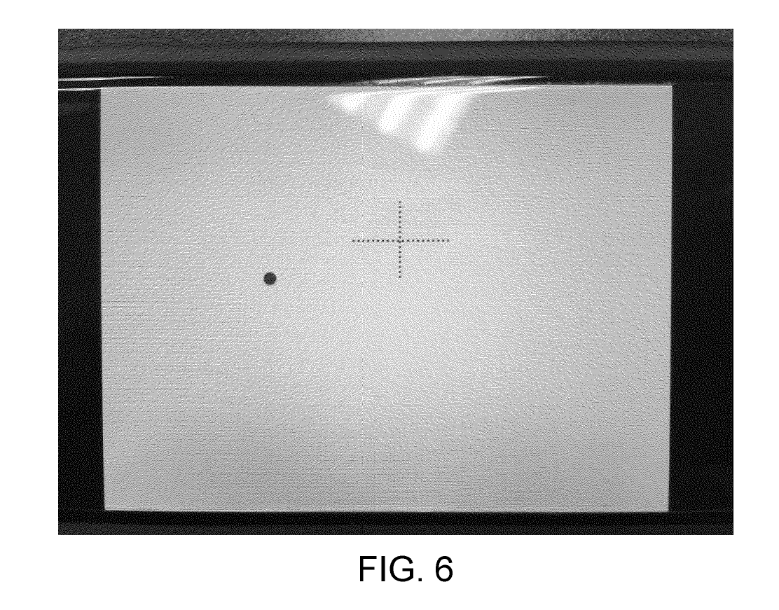

To test a subject, the smartphone 108 display within the virtual reality headset 106 generates a visual stimulus, which is displayed to each eye of the subject in a virtual reality environment. In some embodiments, the virtual reality headset 106 may be capable of generating and displaying the visual stimulus to the subject by itself. In some instances, the portable concussion tester 104, in addition to providing the visual stimulus, is also capable of providing one or more of a sound, touch, and sensory stimulus for testing purposes. The subject may be instructed to perform a task with respect to one or more provided stimulus. For example, the stimulus may be a movable dot and a fixation target, as shown in FIG. 6 and the subject is instructed to move the dot over the fixation target with head movements. Other types of subject tasks or responses with respect to a stimulus (e.g., audio, visual, optical, etc.)

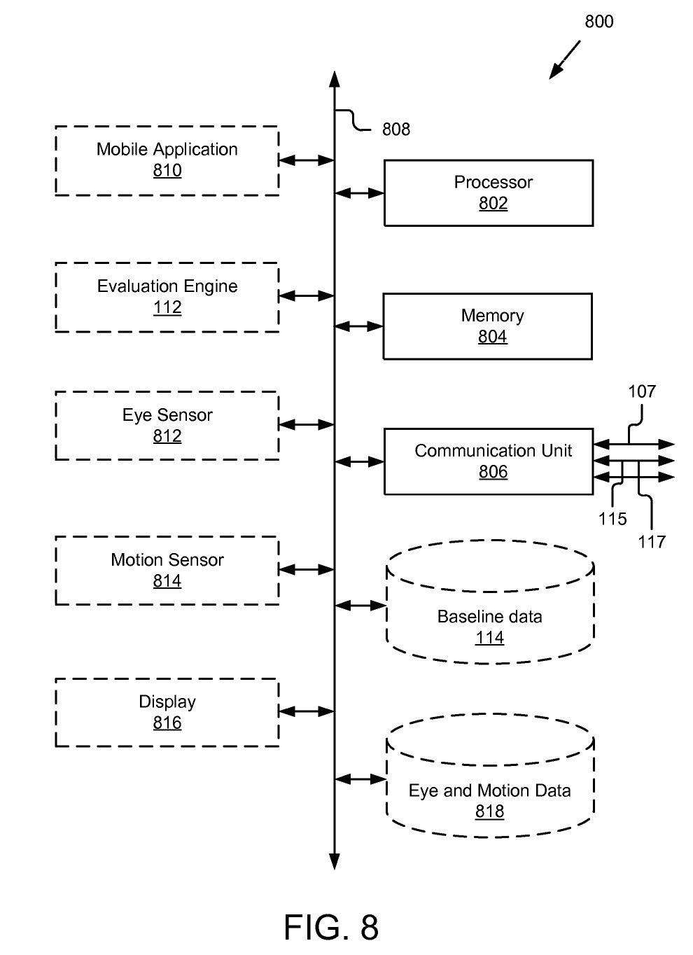

may include, for example, pushing a button or a mechanical clicker, verbal response, doing a head nod, and/or any other body motion. Timing and accuracy of the subject's responses or reactions to a given task are recorded and then evaluated to assess the subject's neurological condition. In a preferred embodiment, the virtual reality headset 106 may include an eye sensor, such as eye sensor 812 (see FIG. 8) and the smartphone 108 may include a motion sensor, such as motion sensor 814 (see FIG. 8), and the virtual reality headset 106 and the smartphone 108 work together or in cooperation to track eye movement (e.g., eye motion, gaze change) and body movement (e.g., head, neck, etc.), respectively while the subject is performing the task. However, it should be understood that the present disclosure is not limited to this configuration and that other configurations are also possible and are within the scope of the present disclosure. Some of the other configurations may include for example, 1) the virtual reality headset 106 includes both the eye sensor 812 as well as the motion sensor 814 for eye and body movements, respectively, 2) the smartphone 108 includes both the eye sensor 812 and the motion sensor 814, and 3) the virtual reality headset 106 includes the motion sensor 814 while the smartphone 108 includes the eye sensor 812.

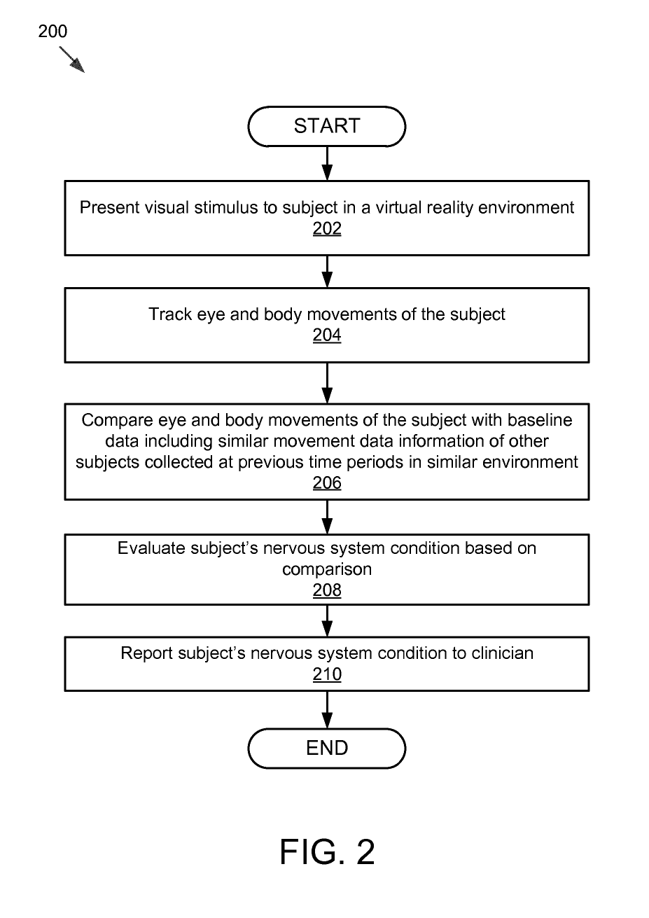

Once the eye and body movement data are obtained using the eye sensor 812 and motion sensor 814 discussed above, the smartphone 108 may perform the evaluation of the subject's condition either locally or remotely with the help of the evaluation server 110. To perform the evaluation locally, the processor of the smartphone 108 (e.g., processor 802) may compare the eye and body movement data of the subject with baseline, normative, or reference data (e.g., the baseline data 114 shown in FIG. 2) stored in the memory of the smartphone 108 (e.g., memory 804). The baseline data 114 include eye and movement data of other subjects who were tested under similar conditions like the subject under test and were verified as having healthy or normal nervous system condition. In some instances, eye and body movement data of the subject discussed herein or any subject upon testing can be stored in the baseline data store 114 that may be used along with already stored eye and movement data of other subjects for future evaluations and/or statistical references or comparisons. In some embodiments, the eye and body movement data of the subject can be evaluated or processed by means other than baseline comparison. For example, by checking 1) synchronicity of eye movements between both eyes, 2) synchronicity of eye and head movement, 3) smoothness of either head or eye movement, 4) delay in subject's reaction or response to displayed stimulus, etc. In case the evaluation is not performed locally, the smartphone 108 then acts as a gateway to relay the eye and body movement data via the network 102 (e.g., WiFi, cellular data network, etc.) to the evaluation server 110 for it to perform the evaluation as discussed in further detail below.

The evaluation server 110 can be a hardware server that includes a processor (e.g., processor 802 (see FIG. 8)), a memory (e.g., memory 804 (see FIG. 8)), and network communication capabilities. The evaluation server 110 is coupled to the network 102 via signal line 115 for communication and cooperation with the portable concussion tester 104 (including the virtual reality headset 106 and the smartphone 108) and one or more clinician devices 116. For instance, the evaluation server 110 communicates with the smartphone 108 via the network 102 to receive the eye and body movement data for evaluation, evaluates the condition of the subject's nervous system, and sends results of the evaluation via the network 102 to the one or more clinician device 116 for display. The evaluation server 110 may perform this evaluation using the processor 802 (see FIG. 8). Although one evaluation server 110 is shown, persons of ordinary skill in the art will recognize that multiple servers can be utilized, either in a distributed architecture or otherwise. For the purpose of this application, the system configuration and operations performed by the system are described in the context of a single evaluation server 110.

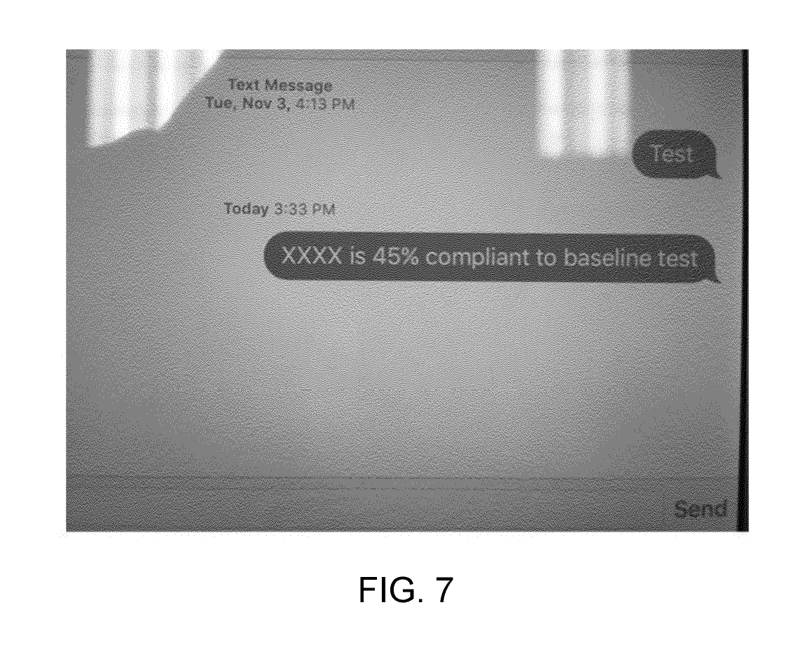

As depicted, the evaluation server 110 may include an evaluation engine 112 for evaluating the condition of the subject's nervous system based on the eye and body movement data recorded by the portable concussion tester 104. The evaluation engine 112 may receive the eye and body movement data from the smartphone 108 via the network 102 as an input and then perform its evaluation thereon. To perform the evaluation, the evaluation engine 112 compares the eye and body movement data of the subject to an expected reaction in a time and position based sequence from either a prerecorded healthy test baseline or from a statistical sample size, which are stored as baseline data 114 in the evaluation server 110. The amount of compliance to this expected sequence is a simple means of a possible net result, which can be expressed in terms of a percentage and can serve a user (e.g., clinician/non-clinician) as status indicator. The evaluation engine 112 may send the computed net result to a clinician device 116 for display. For instance, the evaluation engine 112 may send percentage compliance of the subject to baseline test as a message to the clinician's smartphone 116a, as shown for example in FIG. 7. Although not shown, it should be noted that the evaluation engine 112 may send results of its evaluation (e.g., compliance message) to any device, such as a smartphone, desktop, smartwatch, tablet, etc. and any user (whether a clinician or a non-clinician) associated with such a device can be able to see and interpret these results. In some embodiments, the results of the evaluation may be adjusted (e.g., simplified, confidential or sensitive information removed, etc.) for a non-clinician user.

The clinician device(s) 116 (any or all of 116a through 116n) are computing devices having data processing and data communication capabilities. The clinician devices 116a through 116n are communicatively coupled to the network 102 via signal lines 117a through 117n respectively to receive results of the evaluation from the evaluation server 110 and display them to their respective users. In some embodiments, a clinician device 116 is a smartphone (indicated by reference numeral 116a), a laptop computer (as indicated by reference numeral 116n), or any of a desktop computer, a netbook computer, a tablet, smartwatch, etc.

Example Methods

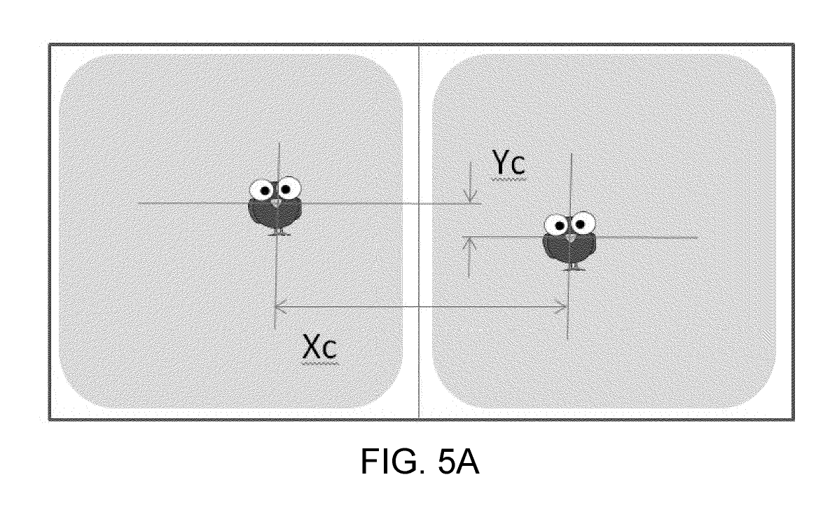

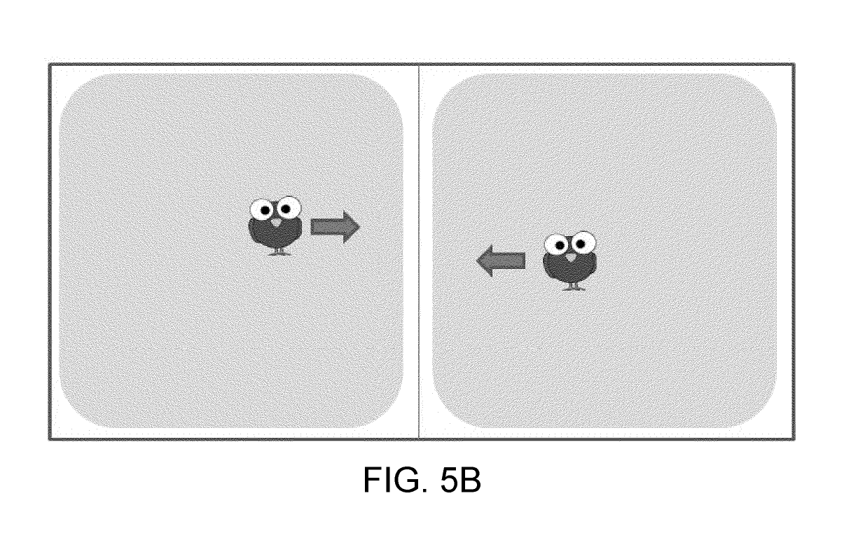

FIG. 2 is a flowchart of an example method 200 for locally testing and evaluating a condition of a subject's nervous system using virtual reality technology according to one aspect of the present invention. It should be understood that locally testing and evaluating the condition means that all the processing is done client-side (i.e., portable concussion tester 104) without involving a server, such as the evaluation server 110. The method 200 begins by first presenting a visual stimulus to the subject in a virtual reality environment 202. The visual stimulus may be displayed separately to each eye of the subject using the virtual reality headset 106 and the smartphone 108. For example, with reference to FIG. 5A, a subject wearing the virtual reality headset 106 will see a bird (visual stimulus) in front of each eye displayed by the screen of the smartphone 108. Next, the subject may be asked to perform a task with respect to the displayed visual stimulus. Some of the exemplary tasks may include 1) following the bird (shown in FIG. 5A) with head and gaze movements, 2) walking on a virtual straight line projected to the subject, 3) moving a dot over a fixation pattern (see FIG. 6) with head movements, and 4) trying to follow two targets (e.g. birds) as they move towards each other (see FIG. 5B), which is one way of testing the nasal field of stereopsis.

Next, in block 204, while the subject is performing a given task, his/her eye and body (e.g., head, neck, etc.) movements are tracked by the eye sensor 812 (see FIG. 8) and the motion sensor 814, respectively. Then, in block 206, the captured eye and body movements of the subject are compared with the baseline data 114 that includes similar movement data information of other subjects who were tested at a previous time in a similar virtual reality type environment. These other subjects, upon testing, were identified and/or verified as having a healthy brain condition. In certain instances, baseline data 114 may further include movement data information of some non-healthy subjects or subjects that were tested under similar environment and/or conditions. The data of these non-healthy subjects can be compared in the same way like that of the healthy subjects discussed elsewhere herein. Comparing eye and body movements of the subject undergoing test with the movement data information of some non-healthy subjects can identify certain other conditions of the subject that may be difficult to identify using data of the healthy subjects. In block 208, the nervous system condition of the subject under testing is evaluated based on the comparison performed in block 206. For instance, based on the comparison with other subjects, a degree of compliance of the subject's condition with the other subjects' condition is determined. If the degree of compliance of the subject is determined to be lower than a certain pre-defined threshold, the subject's condition is evaluated as critical. Otherwise, the subject's condition is evaluated as healthy or normal. Finally, in block 210, the subject's nervous system condition is reported to a clinician for further analysis thereof. In some embodiments, the operations discussed with respect to blocks 202 and 204 are performed by the virtual reality headset 106 in conjunction with the smartphone 108 while the operations with respect to blocks 206-210 are performed by the processor of the smartphone 108.

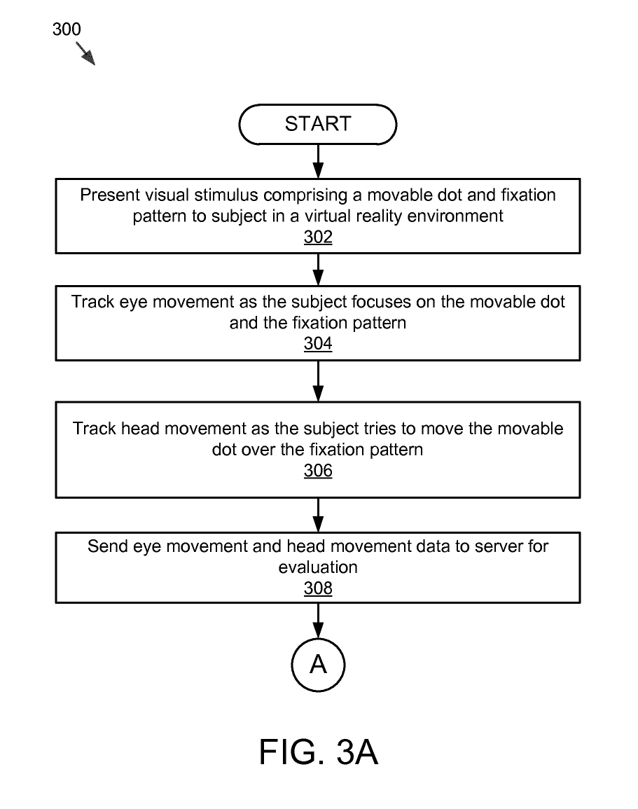

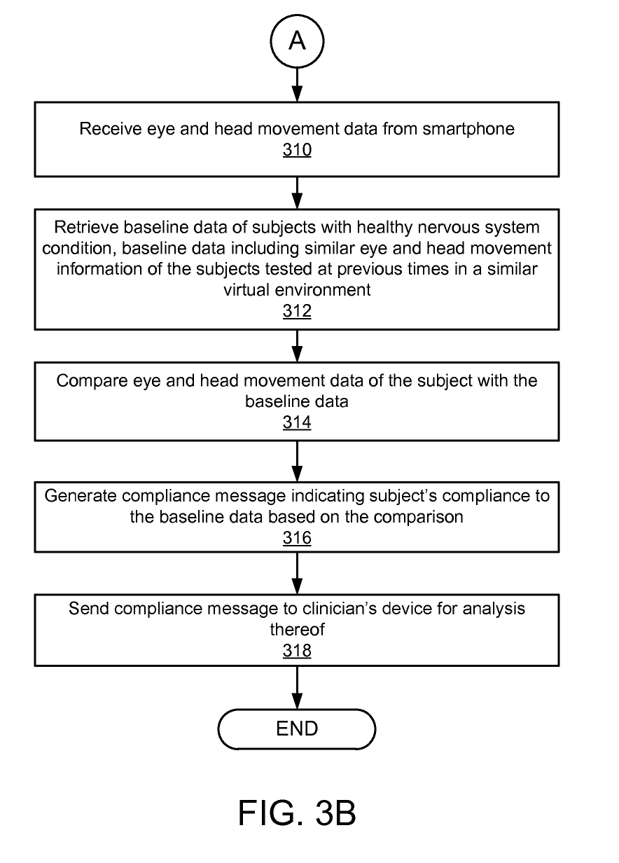

FIGS. 3A and 3B are flowcharts of an example method 300 for testing a subject and remotely evaluating a condition of a subject's nervous system according to one aspect of the present invention. It should be understood that a particular visual stimulus and a task with respect to that stimulus is described in this method 300. However, the method 300 is not limited to such a case and that a variety of other stimulus (e.g., sound, sensory, touch, etc. in addition to visual) and tasks are also possible and are within the scope of this disclosure. The method 300 begins by first presenting 302 a visual stimulus comprising a movable dot and a fixation pattern (see FIG. 6) in a virtual reality environment using the portable concussion tester 104. The subject may be instructed to move the movable dot over the fixation pattern using head movements. In block 304, movements of both eyes of the subject are tracked by the eye sensor 812 (see FIG. 8) as the subject focuses on the movable dot and the fixation pattern. Next, in block 306, head movements are tracked by the motion sensor 814 (see FIG. 8) as the subject tries to move the dot over the fixation pattern by moving his/her head in the direction of the fixation pattern. In some instances, the eye sensor 812 is built-in the virtual reality headset 106 and the motion sensor 814 is built-in the smartphone 108 as discussed elsewhere herein.

For the purposes of remote evaluation, the smartphone 108 may act like a gateway between the virtual reality headset 106 and evaluation server 110. In block 308, the smartphone 108 sends the eye and head movement data (captured in blocks 304 and 306) to the evaluation server 110 using standard communication protocols such as WiFi, cellular networks, or any other means of transmission known in the art.

Referring to FIG. 3B, it should be understood that the operations will now be discussed from the server-side. In block 310, the evaluation server 110 receives the eye and head movement data from the smartphone 108 via a network, such as the network 102. In block 312, the evaluation engine 112 of the server 110 retrieves baseline data 114 of subjects with healthy nervous system condition. The baseline data 114 include eye and head movement information of these subjects who were tested at a previous time using the same visual stimulus and task discussed herein. Next, in block 314, the evaluation engine 112 compares the eye and head movement data of the subject with the baseline data 114. Based on the comparison, the evaluation engine 112, in block 316, generates a compliance message indicating subject's compliance to the baseline data of other subjects, as shown in FIG. 7. Finally, in block 318, the evaluation server 110 sends the compliance message to a clinician mobile device, such as clinician's smartphone 116a for analysis thereon.

Example Computing Device

FIG. 8 is a block diagram of an example computing device 800, which may be representative of a computing device included in the portable concussion tester 104, the evaluation server 110, and/or the clinician device 116. As depicted, the computing device 800 may include a processor 802, a memory 804, a communication unit 806, a mobile application 810, the evaluation engine 112, an eye sensor 812, a motion sensor 814, a display 816, baseline data 114, and eye and motion data 818, which may be communicatively coupled by a communication bus 808. The bus 808 can include a conventional communication bus for transferring data between components of a computing device or between computing devices.

Depending upon the configuration, the computing device 800 may include differing or some of the same components. For instance, in the case of portable concussion tester 104 (combining both the virtual reality headset 106 and the smartphone 108 as one unit), the computing device 800 may include the processor 802, the memory 804, the communication unit 806, the mobile application 810, the eye sensor 812, the motion sensor 814, the display 816, the baseline data 114, and the eye and motion data 818. In the case of the evaluation server 110, the computing device 800 may include the components 802, 804, 806, 112, 114, and 818. In the case of the clinician device 116, the computing device 800 may include the components 802, 804, 806, 810, and 816. It should be understood that the above configurations are provided by way of example and numerous further configurations are contemplated and possible.

The processor 802 may execute various hardware and/or software logic, such as software instructions, by performing various input/output, logical, and/or mathematical operations. The processor 802 may have various computing architectures to process data signals including, for example, a complex instruction set computer (CISC) architecture, a reduced instruction set computer (RISC) architecture, and/or architecture implementing a combination of instruction sets. The processor 802 may be physical and/or virtual, and may include a single core or plurality of processing units and/or cores. In some embodiments, the processor 802 may be capable of generating and providing electronic display signals to a display device, such as the display 816, supporting the display of images, capturing and transmitting images, performing complex tasks including various types of feature extraction and sampling, etc. In some embodiments, the processor 802 may be coupled to the memory 804 via a data/communication bus to access data and instructions therefrom and store data therein. The bus 808 may couple the processor 802 to the other components of the computer device 800.

The memory 804 may store instructions and/or data that may be executed by the processor 802. In some embodiments, the memory 804 stores at least the mobile application 810, the evaluation engine 110, the baseline data 114, and the eye and motion data 818. In some embodiments, the memory 804 may also be capable of storing other instructions and data including, for example, an operating system, hardware drivers, other software applications, databases, etc. The memory 804 is coupled to the bus 808 for communication with the processor 802 and other components of the computing device 800. The memory 804 may include a non-transitory computer-usable (e.g., readable, writeable, etc.) medium, which can be any apparatus or device that can contain, store, communicate, propagate or transport instructions, data, computer programs, software, code, routines, etc. for processing by or in connection with the processor 802. A non-transitory computer-usable storage medium may include any and/or all computer-usable storage media. In some embodiments, the memory 804 may include volatile memory, non-volatile memory, or both. For example, the memory 804 may include a dynamic random access memory (DRAM) device, a static random access memory (SRAM) device, flash memory, a hard disk drive, a floppy disk drive, a CD ROM device, a DVD ROM device, a DVD RAM device, a DVD RW device, a flash memory device, or any other mass storage device known for storing instructions on a more permanent basis.

In some embodiments, one or more of the portable concussion testers 104, the evaluation server 110, and the one or more clinician devices 116 are located at the same or different locations. When at different locations, these components may be configured to communicate with one another through a wired and/or wireless network communication system, such as the communication unit 806. The communication unit 806 may include network interface devices (I/F) for wired and wireless connectivity. For example, the communication unit 806 may include a CAT-type interface, USB interface, or SD interface, transceivers for sending and receiving signals using Wi-Fi™, Bluetooth®, or cellular communications for wireless communication, etc. The communication unit 806 may be coupled to the network 102 via the signals lines 107, 115, and 117. The communication unit 806 can link the processor 802 to a computer network, such as the network 102 that may in turn be coupled to other processing systems.

The mobile application 810 is storable in the memory 804 and executable by the processor 802 of a clinician device 116 and/or the smartphone 108 to provide for user interaction, receive user input, present information to the user via the display 816 and send data to and receive data from the other entities of the system 100 via the network 102. In some embodiments, the mobile application 810 may generate and present user interfaces based at least in part on information received from the evaluation server 110 via the network 102. For example, a user/clinician may use the mobile application 810 to receive results of an evaluation computed by the evaluation server 110 on his/her clinician device 116. In some embodiments, the mobile application 810 includes a web browser and/or code operable therein, a customized client-side application (e.g., a dedicated mobile app), a combination of both, etc.

The eye sensor 812 and the motion sensor 814 are sensors for tracking eye and body movements of a user/subject, respectively. In some instances, the eye sensor 812 is a CCD camera that is capable of tracking both the eyes of the user simultaneously. The motion sensor 814 may be a gyro sensor and/or an accelerometer that is capable of tracking body movements including, but not limited to, head, neck, hands, and/or feet, etc. movements of the user. In some embodiments, the eye sensor 812 and/or the motion sensor 814 may be coupled to the components 802, 804, 806, 810, 112, and 818 of the computing device 800 via the bus 808 to send/or receive data.

The display 816 represents any device equipped to display electronic images and data as described herein. The display 816 may be any of a conventional display device, monitor or screen, such as an organic light-emitting diode (OLED) display, a liquid crystal display (LCD). In some embodiments, the display 816 is a touch-screen display capable of receiving input from one or more fingers of a user. For example, the device 816 may be a capacitive touch-screen display capable of detecting and interpreting multiple points of contact with the display surface.

The baseline data 114 and the eye and motion data 818 are information sources for storing and providing access to data. The baseline data 114 include eye and motion data of one or more subjects who were tested for their nervous system condition evaluation using the virtual reality technology discussed herein and were identified as having a healthy or normal condition. In some embodiments, the baseline data 114 may further include prior eye and motion data of the subject currently under evaluation or testing whose prior data may be used to assess a trend, change, and/or progression in the subject's neurological condition. The eye and motion data 818 include eye and body (e.g., head, neck, etc.) movements of a subject undergoing test that are captured as the subject performs a task with respect to a visual stimulus displayed to the subject inside the virtual reality headset 106. For example, a subject is instructed to focus on a dot and as the dot moves, the subject's eye and head movements are recorded with respect to the moving dot and are stored as the eye and motion data 818. The baseline data 114 and the eye and motion data 818 may be stored in the evaluation server 110 for remote evaluation or may be stored in the memory 804 of the smartphone 108 for local evaluation as discussed elsewhere herein. In some embodiments, the baseline data 114 and the eye and motion data 818 may be coupled to the components 802, 804, 806, 810, 112, 812, and 814 of the computing device 800 via the bus 808 to receive and provide access to data. The baseline data 114 and the eye and motion data 818 can each include one or more non-transitory computer-readable mediums for storing the data. In some embodiments, the baseline data 114 and the eye and motion data 818 may be incorporated with the memory 804 or may be distinct therefrom.

In the above description, for purposes of explanation, numerous specific details are set forth in order to provide a thorough understanding of the specification. It should be apparent, however, that the subject matter of the present application can be practiced without these specific details. It should be understood that the reference in the specification to “one embodiment”, “some embodiments”, or “an embodiment” means that a particular feature, structure, or characteristic described in connection with the embodiment is included in one or more embodiments of the description. The appearances of the phrase “in one embodiment” or “in some embodiments” in various places in the specification are not necessarily all referring to the same embodiment(s).

Furthermore, the description can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

The foregoing description of the embodiments of the present subject matter has been presented for the purposes of illustration and description. It is not intended to be exhaustive or to limit the present embodiment of subject matter to the precise form disclosed. Many modifications and variations are possible in light of the above teaching. It is intended that the scope of the present embodiment of subject matter be limited not by this detailed description, but rather by the claims of this application. As will be understood by those familiar with the art, the present subject matter may be embodied in other specific forms without departing from the spirit or essential characteristics thereof. Furthermore, it should be understood that the modules, routines, features, attributes, methodologies and other aspects of the present subject matter can be implemented using hardware, firmware, software, or any combination of the three.

1. A system for testing a condition of a subject's nervous system using virtual reality technology, said system comprising:
a virtual reality headset for displaying a visual stimulus separately to each eye of the subject in a virtual reality environment;
an eye sensor for tracking eye movements as the subject focuses on the visual stimulus;
a motion sensor for tracking body movements as the subject focuses on the visual stimulus in the virtual reality environment; and
a processor for evaluating the condition of the subject's nervous system based on the eye and the body movements, wherein results of the evaluation are stored or transmitted for display to a user.

2. The system as recited in claim 1, wherein the processor evaluates the condition of the subject's nervous system by comparing the eye and the body movements of the subject with baseline data that include one or more of 1) eye and body movement information of other subjects with healthy nervous system condition who were tested under similar virtual environment and 2) prior eye and body movement information of the subject.

3. The system as recited in claim 2, wherein the eye and body movement information of the other subjects include one or more expected parameters and evaluating the condition of the subject's nervous system includes computing compliance with the one or more expected parameters.

4. The system as recited in claim 3, wherein the one or more expected parameters include one or more of average reaction time, gaze accuracy, and smoothness of gaze and head movement to the visual stimulus.

5. The system as recited in claim 3, wherein the results of the evaluation are transmitted by the processor as a message to a user's device.

6. The system as recited in claim 1, wherein the user is a clinician or a person responsible for evaluating a neurological condition.

7. The system as recited in claim 2, wherein the eye sensor is attached to or embedded in the virtual reality headset.

8. The system as recited in claim 2, wherein the motion sensor is embedded in a smartphone.

9. The system as recited in claim 8, wherein the virtually reality headset comprises the smartphone and the visual stimulus is displayed by a screen of said smartphone.

10. The system as recited in claim 9, wherein the smartphone and the virtual reality headset are locally connected with each other.

11. The system as recited in claim 9, wherein the processor is included in the smartphone and the baseline data is stored in the memory of the smartphone for locally evaluating the condition of the subject's nervous system.

12. The system as recited in claim 9, wherein the smartphone acts as a gateway to send the eye and body movement data to a server for remote evaluation, wherein the processor is the server's processor and the baseline data is stored at the server.

13. The system as recited in claim 1, wherein the body movements are head movements.

14. The system as recited in claim 1, wherein the eye sensor is capable of tracking both eyes of the subject simultaneously.

15. The system as recited in claim 1, wherein the subject is instructed to perform a task with respect to the visual stimulus, said task comprising one of 1) following an object as it moves in the virtual reality environment, 2) walking on a virtual line, and 3) superimposing a movable dot over a fixation target.

16. The system as recited in claim 15, wherein the subject is instructed to superimpose the movable dot over the fixation target by head movements which are tracked by the motion sensor.

17. The system as recited in claim 9, wherein the virtual reality headset comprising the smartphone is further capable of providing one or more of a sound, touch, and any sensory stimulus in addition to the visual stimulus.

18. The system as recited in claim 1, wherein the visual stimulus is displayed to one eye at a time to the subject and difference in subject's behavior based on displaying the visual stimulus to either eye versus displaying the stimulus to both the eyes simultaneously is compared for the purpose of evaluation.

19. The system as recited in claim 1, wherein the visual stimulus that is displayed separately to each eye is moved towards each other to identify the nasal field of stereopsis.

20. The system as recited in claim 1, wherein the eye sensor is a CCD camera and the system further comprises an infrared or visible light illumination source for making the eye visible to the camera.

21. The system as recited in claim 1, wherein said system is used to test one or more of 1) a concussion or traumatic brain injury test, 2) a driving under the influence (DUI) or driving while intoxicated (DWI) test, and 3) Alzheimer's, dementia and other age related disease states.

22. A method for testing a condition of a subject's nervous system using virtual reality technology, said method comprising:
displaying a visual stimulus to the subject in a virtual reality environment;
tracking eye and body movements of the subject as the subject focuses on the visual stimulus;
evaluating the subject's nervous system condition based on the eye and body movements of the subject; and
reporting results of the evaluation describing the subject's nervous system condition to a user for further analysis thereof

23. The method as recited in claim 22, wherein evaluating the subject's nervous system condition comprises comparing the eye and the body movements of the subject with baseline data that include one or more of 1) eye and body movement information of other subjects with healthy nervous system condition who were tested under similar virtual environment and 2) prior eye and body movement information of the subject.

24. The method as recited in claim 23, wherein the eye and body movement information of the other subjects include one or more expected parameters and evaluating the condition of the subject's nervous system includes computing compliance with the one or more expected parameters.

25. The method as recited in claim 24, wherein the one or more expected parameters include one or more of average reaction time, gaze accuracy, and smoothness of gaze and head movement to the visual stimulus.

26. The method as recited in claim 22, wherein the body movements are head movements.

27. The method as recited in claim 22, wherein the subject is instructed to perform a task with respect to the visual stimulus, said task comprising one of 1) following an object as it moves in the virtual reality environment, 2) walking on a virtual line, and 3) superimposing a movable dot over a fixation target.

28. The method as recited in claim 27, wherein the subject is instructed to superimpose the movable dot over the fixation target by head movements.

29. The method for as recited in claim 22 further comprising:
providing one or more of a sound, touch, and any sensory stimulus in addition to displaying the visual stimulus.

30. The method as recited in claim 22, wherein the user is a clinician or a person responsible for evaluating a neurological condition.
